(19) United States
(12) Patent Application Publication
(10) Pub. No.: US 2013/0282068 A1
(43) Pub. Date: Oct. 24, 2013

(54) AED TREATMENT RECOMMENDATION METHOD AND DEVICE

(71) Applicants: Mustafa H. Sagiroglu, Bellevue, WA (US); James Walter Taylor, Laguna Niguel, CA (US)

(72) Inventors: Mustafa H. Sagiroglu, Bellevue, WA (US); James Walter Taylor, Laguna Niguel, CA (US)

(21) Appl. No.: 13/452,476

(22) Filed: Apr. 20, 2012

(51) Int. Cl. A61N 1/39; A61M 16/00

(52) U.S. Cl. 607/3; 607/7

(57) ABSTRACT

A device and method for recommending an initial treatment of either a defibrillation shock or CPR for a cardiac arrest victim. An embodiment of the invention is directed to an AED with treatment protocols selected from a set of external defibrillation, CPR, or a combination thereof. The AED includes a power generation circuit, pair of external electrodes, and an ECG sensor. AED further includes a control system including a microprocessor configured to determine a survivability index number for a patient and recommend a treatment protocol using the AED as well as a communication system configured to communicate the selected protocol to a user of the AED.

The present invention relates to improved methods and apparatus involving the integrated use of Automated External Defibrillators (AEDs) and cardiopulmonary resuscitation (CPR). Specifically, this invention relates to AEDs and methods for automatically making a determination of the appropriate therapy to treat a cardiac arrest victim, such as CPR or a defibrillation pulse.

### BACKGROUND OF THE INVENTION

Cardiac arrest is widely-understood to be a substantial public health problem and a leading cause of death in most areas of the world. Each year in the U.S. and Canada, approximately 350,000 people suffer a cardiac arrest and receive attempted resuscitation. Accordingly, the medical community has long sought ways to more successfully treat cardiac arrest victims through CPR and application of defibrillation shocks to restore a normal heart rhythm to persons experiencing this type of event. Automatic External Defibrillators were first developed decades ago to help treat incidents of cardiac arrest. Since their creation, AEDs have become prevalent in public locales such as offices, shopping centers, stadiums, and other areas of high pedestrian traffic. AEDs empower citizens to provide medical help during cardiac emergencies in public places where help was previously unavailable in the crucial early stages of a cardiac event.

Fully automated external defibrillators capable of accurately detecting ventricular arrhythmia and non-shockable supraventricular arrhythmia, such as those described in U.S. Pat. No. 5,474,574 to Payne et al., have been developed to treat unattended patients. These devices treat victims suffering from ventricular arrhythmias and have high sensitivity and specificity in detecting shockable arrhythmias in real-time. Further, AEDs have been developed to serve as diagnostic monitoring devices that can automatically provide therapy in hospital settings, as exhibited in U.S. Pat. No. 6,658,290 to Lin et al.

Most of the AEDs available today attempt to classify ventricular rhythms and distinguish between shockable ventricular rhythms and all other rhythms that are non-shockable. This detection and analysis of ventricular rhythms provides some real-time analysis of ECG waveforms. The functionality, accuracy and speed of a particular AED heavily depends on the algorithms and hardware utilized for analysis of ECG waveforms. In many implementations, the algorithms used in AEDs depend on heart rate calculations and a variety of morphology features derived from ECG waveforms, like ECG waveform factor and irregularity as disclosed in U.S. Pat. No. 5,474,574 to Payne et al. and U.S. Pat. No. 6,480,734 to Zhang et al. Further, in order to provide sufficient processing capability, current AEDs commonly embed the algorithms and control logic into microcontrollers.

As advances have taken place in the field of AEDs, there have been significant medical advancements in the understanding of human physiology and how it relates to medical care. These advancements in medical research have lead to the development of new protocols and standard operating procedures in dealing with incidents of physical trauma. For example, in public access protocols for defibrillation, recent guidelines have emphasized the need for the use of both CPR and AEDs and suggested an inclusive approach involving defibrillation integrated with CPR. Despite advances in AED technology, many current AEDs are not fully able to implement the current medically suggested methods of integrated CPR and AED use.

A challenge that AEDs designs now face involves how to effectively integrate the new guidelines for treatment and appropriately take into account the medical needs of various patients. Most current AEDs employ a “shock first” strategy in which the AEDs recommend that a shock be delivered to a victim before attempting CPR. While this methodology may be correct in a majority of circumstances, there are situations where it is most beneficial to implement CPR before a shock. Unfortunately, current AEDs are unable to or inefficient at detecting what the most beneficial initial treatment should be for a cardiac arrest victim. Therefore, improved methods and apparatus for gathering and analyzing ECG signal data and communicating the best initial treatment of a cardiac arrest victim are desired.

### SUMMARY OF THE INVENTION

The present invention provides a device and method for recommending an initial treatment protocol for a cardiac arrest victim. In most circumstances, this includes whether to first administer a defibrillation shock or CPR. Improvements to the integrated use of CPR and AEDs is made possible by the various embodiments of the methods and apparatus of the present invention such that an appropriate “shock first” or “CPR first” rescue protocol is efficiently and accurately advised.

One embodiment of the invention is directed to an AED with treatment protocols selected from a set of external defibrillation, CPR, or a combination thereof. The AED includes a power generation circuit that provides power for treating a shockable heart rhythm with a defibrillation pulse, a pair of external electrodes adapted for delivering a defibrillation pulse, an ECG sensor that obtains an ECG signal corresponding to patient heart activity, and a communication system for communicating a treatment protocol. The AED also includes a control system including a microprocessor configured to determine a survivability index number for a patient and recommend and/or implement a treatment protocol in response to the survivability index number. Specifically, determining the survivability index number includes transforming the ECG signal data into first derivative velocity domain ECG signal data, sampling the velocity domain ECG signal data, sorting the velocity domain ECG signal data samples into one or more groups based on the value of the ECG signal data samples wherein the value stored in each group corresponds to the number of samples sorted into that group, and obtaining a distribution density of the values stored in one or more groups.

Another embodiment of the invention is directed to a method of automatically determining an appropriate treatment for a cardiac arrest victim with an AED. The method includes obtaining ECG signal data from the cardiac arrest victim, transforming the ECG signal data into first derivative velocity domain ECG signal data, and determining a survivability index number for the cardiac arrest victim. Determining a survivability index number includes sampling the velocity domain ECG signal data, sorting the velocity domain ECG signal data samples into one or more groups based on the value of the ECG signal data samples wherein the value stored in each group corresponds to the number of samples sorted into that group, and obtaining a distribution density of the values stored in one or more of the groups. The method also includes determining a treatment protocol for the cardiac arrest victim based on the survivability index number, wherein the treatment protocol is selected from a set of: external defibrillation, CPR or a combination of external defibrillation and CPR. The method finally includes either or both of communicating the treatment protocol and/or implementing the treatment protocol.

The current disclosure recognizes that part of the difficulty for current AEDs in recommending the appropriate initial treatment lies in the processing of the ECG signal. For example, current techniques generally revolve around analysis of the frequency spectrum of the ECG signal. However, analyzing the frequency components of the ECG signal can be computation intensive and affected by significant amounts of noise. Further, many current algorithms are generally unable to effectively distinguish among patients of intermediate down time as current approaches do not offer a stable monotonic behavior with the duration of downtime or offer a clean separation of non-VF and noise classes from treatable VF rhythms. Accordingly, the apparatus and method in this disclosure have been contemplated in recognition of these deficiencies and provide an improvement to these and other past techniques.

In some embodiments, the device includes a pair of electrodes that are adapted to be connected to a cardiac arrest victim when the device is in use. The electrodes may conduct electrical signals from the victim to the device and, conversely, from the device to the victim. In at least one embodiment, the signals coming from the victim are ECG signals and the therapy coming from the device includes high energy defibrillation pulses.

In one illustrative embodiment, the device includes an ECG signal filter and amplifier connected to the pair of electrodes. To a large extent, the filter filters out signals other than the ECG signal and the amplifier amplifies the ECG signal to allow for easier processing of the ECG signal.

In some embodiments, the filter and amplifier are connected to a controller that controls all the elements of the device. The device includes at least a high energy circuit, an ECG signal filter and amplifier, a memory, and a user interface. The controller may be configured to receive input from the ECG signal filter and amplifier. The controller may also be configured to execute an improved initial treatment recommendation module. The recommendation module may use data from the ECG signal to compute a survivability index number. In one illustrative embodiment, the recommendation module transforms the ECG signal data into first derivative velocity domain ECG signal data. The recommendation module may then sample the velocity domain signal data and sort those samples into various groups or “bins” based on the sample value. The value stored in each bin corresponds to the number of samples sorted into that bin. Next, the recommendation module may obtain a distribution density value of the bin values. The recommendation module uses at least the distribution density value to determine a survivability index number.

In other embodiments, the recommendation module may also obtain the number of recognizable beats per minute present in the ECG signal data, if any, and the envelope amplitude of the ECG signal data. Preferably, the recommendation module may then combine the beats per minute, the envelope amplitude, and the distribution density to determine a survivability index number.

In at least one embodiment, the controller uses the determined survivability index number to recommend and/or implement an initial treatment protocol for treating a cardiac arrest victim. The controller may compare the determined survivability index number to predetermined survivability index numbers. The device may contain a user interface, or other device, to communicate the recommended treatment to a user, and/or in the case of a fully automatic external defibrillator, begin implementing the recommended treatment.

Other embodiments may include use of the SI to set the initial shock energy for defibrillation. This initial shock energy may be particularly important due to the relationship between the level of energy necessary for a defibrillation shock and the amount of time that has passed since a cardiac event. Specifically, when applying an initial defibrillation shock to a cardiac arrest victim, the probability of success of a shock is related to both the energy of the shock and inversely related to the length of time since the heart has stopped. Accordingly, using SI to set the initial shock energy may be particularly useful to provide the most effective AED therapy possible.

### BRIEF DESCRIPTION OF THE DRAWINGS

The invention may be more completely understood in consideration of the following detailed description of various embodiments of the invention in connection with the accompanying drawings, in which:

FIG. 1A is an exemplary depiction of the delineation for advice of CPR or defibrillation for the best return to spontaneous circulation (ROSC) outcome.

FIG. 1B shows a rescuer using an AED on a cardiac arrest victim, according to an embodiment of the invention.

FIG. 1C is block diagram of part of an exemplary AED, according to an embodiment of the invention.

FIG. 2 is a flow chart describing a method of calculating a survivability index number, according to an embodiment of the invention.

FIG. 3 is a flow chart describing a method of determining an initial therapy recommendation based on a survivability index number, according to an embodiment of the invention.

FIG. 4 is a flow chart describing a method wherein a survivability index number is repeatedly calculated and the treatment protocol recommendation is repeatedly updated, according to an embodiment of the invention.

FIG. 5 is a flow chart describing a method wherein the survivability index number is used to calculate the amount of shock energy in a defibrillation pulse, according to an embodiment of the invention.

FIG. 6A is an illustrative graph of samples of a two second ECG data segment, according to an embodiment of the invention.

FIG. 6B is an illustrative graph of samples of the first difference “velocity” of the two second ECG data segment of FIG. 6A, according to an embodiment of the invention.

FIG. 6C is an illustrative graph of the corresponding bin distribution of the velocity converted data samples in FIG. 6A and the range for density computation, according to an embodiment of the invention.

FIG. 7A is an illustrative graph of samples of the survivability index for a group of rescue files surrounding the time of shock delivery, according to an embodiment of the invention.

FIG. 7B is an illustrative graph of samples of the survivability index for a group of rescue files for the last fifteen seconds of data, according to an embodiment of the invention.

FIG. 8 is an illustrative graph showing an ROC (Receiver Operating Characteristics) curve showing the sensitivity and specificity of the disclosed method as tested on 240 patient data files.

While the invention is amenable to various modifications and alternative forms, specifics thereof have been shown by way of example in the drawings and will be described in detail. It should be understood, however, that the intention is not to limit the invention to a particular embodiments described. On the contrary, the intention is to cover all modifications, equivalents, and alternatives falling within the spirit and scope of the invention as defined by the appended claims.

### DETAILED DESCRIPTION OF THE DRAWINGS

The invention may be embodied in other specific forms without departing from the essential attributes thereof, therefore, the illustrated embodiments should be considered in all respects as illustrative and not restrictive.

The AHA previously recommended that all rescuers, regardless of training, perform CPR on all cardiac arrest victims, and that chest compressions should be the initial treatment action for all victims regardless of age. This recommendation recognizes that CPR typically improves a victim's chance of survival by providing critical blood circulation in the heart and brain. However, the AHA has now recognized that there are situations where issuing a defibrillation shock from an AED should be the initial treatment for a cardiac arrest victim. Accordingly, the present invention helps meet the need of an AED which provides efficient and automated determination of the appropriate therapy.

The current disclosure relates to apparatus and methods which utilize a defibrillation success predictive algorithm which applies decision rules to an AED therapy decision. The algorithm effectively provides a measure of the state of a patient's cardiac muscle in order to appropriately guide therapy. To do this, a “survivability index” (SI) is calculated relating ventricular fibrillation (VF) waveform parameters to the likelihood of successful defibrillation, that is, a conversion of an electrical rhythm that supports a return to spontaneous circulation (ROSC).

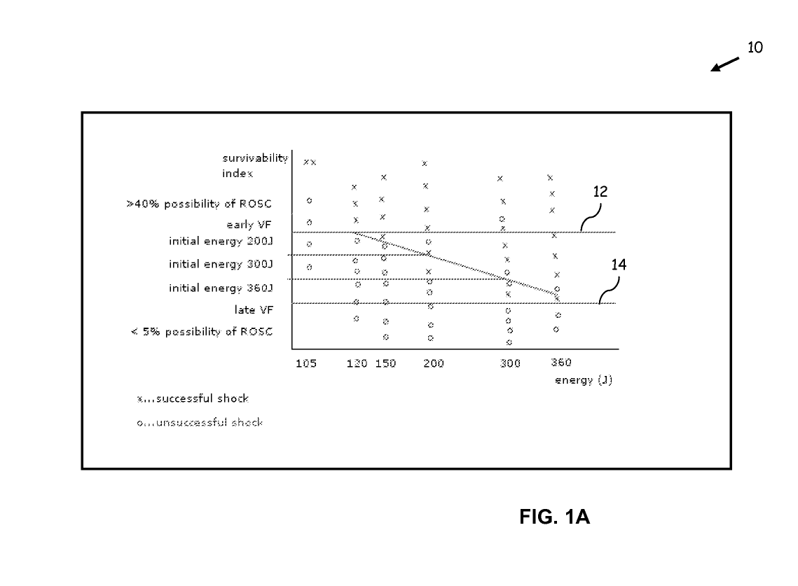

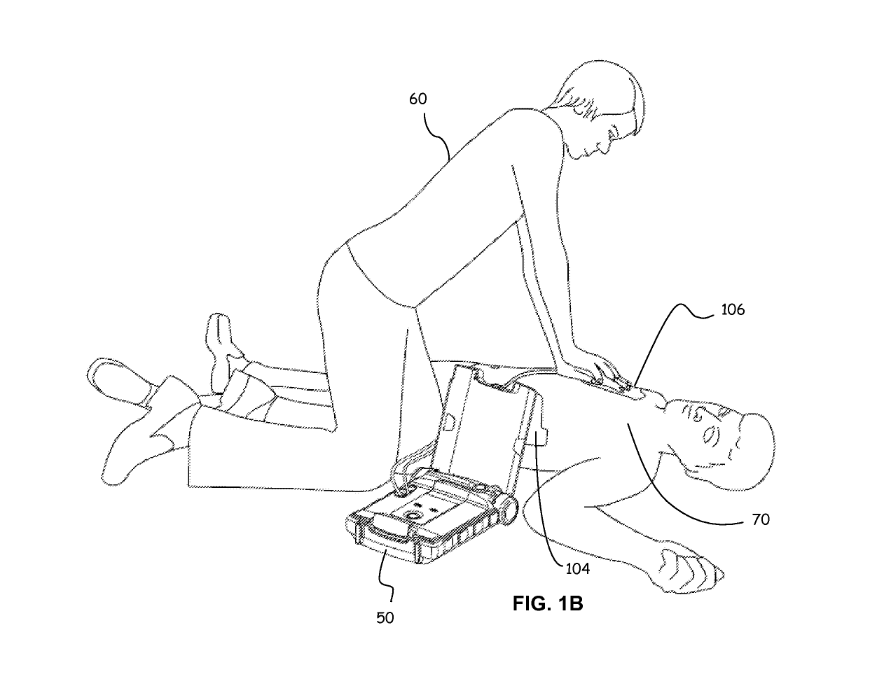

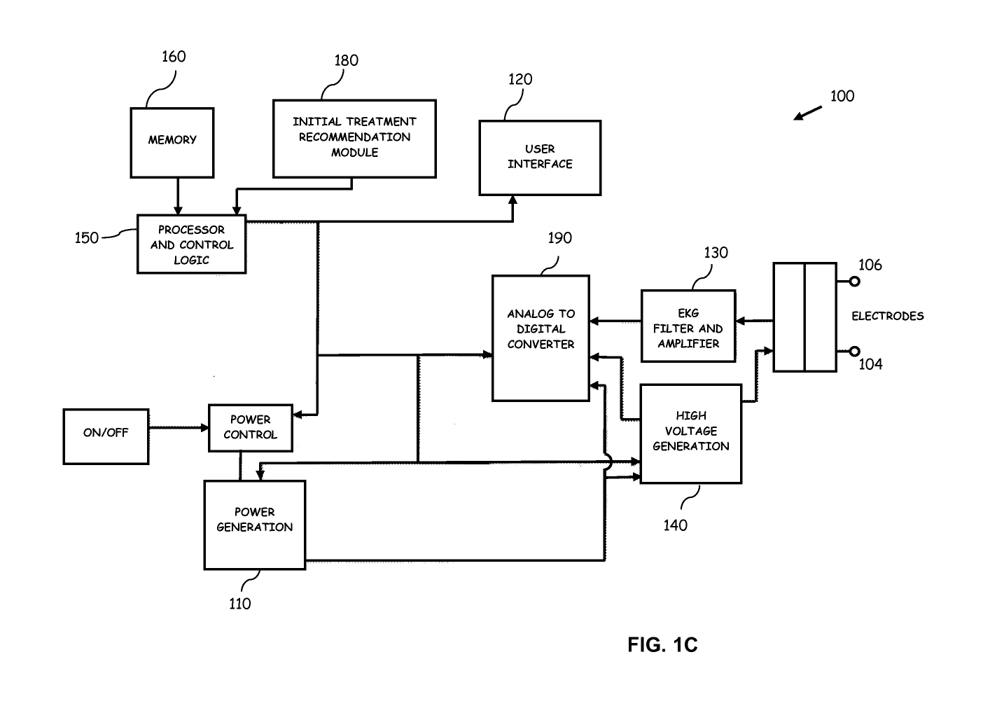

For illustrative purposes, FIG. 1A provides an exemplary depiction of a chart 10 representing the delineation for advice of CPR or defibrillation for the best return to spontaneous circulation (ROSC) outcome. The top line 12 represents the early stages of VF (coarse). The top line 12 also represents the lowest SI limit for which a defibrillation shock with 200 J of energy will produce the highest likelihood (>40%) of ROSC. The lowermost line 14 indicates late VF and the conditions for which CPR should be advised as a first therapy.

As mentioned above, one use of the SI is in the selection of defibrillation or CPR as the appropriate initial rescue therapy. Although the AED is equipped to originally make this determination, in some embodiments the AED could be set to administer a default therapy of either defibrillation or CPR. The algorithm implemented in the AED, however, would be enabled to override the default therapy. For example, a “defibrillate first” default therapy would be overridden if the SI indicates a late VF which would be unlikely to be converted by defibrillation. Similarly, the algorithm implemented in the AED would be enabled to override a “CPR first” therapy protocol if the calculated SI indicates an early VF, for example, that is likely to be successfully converted by defibrillation.

In some embodiments calculation of SI may include using a change in SI during CPR to detect ineffective CPR and to trigger more aggressive CPR prompting. A change in SI may also be used to terminate CPR and to initiate defibrillation when the change in SI indicates the patient is likely to respond to defibrillation.

Other embodiments may include use of the SI to set the initial shock energy for defibrillation. This initial shock energy may be particularly important due to the relationship between the level of energy necessary for a defibrillation shock and the amount of time that has passed since a cardiac event. Specifically, when applying an initial defibrillation shock to a cardiac arrest victim, the probability of success of a shock is related to both the energy of the shock and inversely related to the length of time since the heart has stopped. Accordingly, using SI to set the initial shock energy may be particularly useful to provide the most effective AED therapy possible.

In some embodiments, outliers may be designed to map into extreme ranges of survivability. For example, regular superventricular rhythms would map to the very survivable range, distinct from early VF and noise would map into an interval in non-survivable values, distinct from fine VF.

The methodology behind calculation of SI relies on a variety of factors. However, it is noteworthy that the SI calculation makes use of the shape and the amplitude of patient ECG waveforms and their representation in the first derivative velocity domain as one factor to determine its value. Embodiments of the present invention have recognized that ECG waveforms present unique characteristics in the velocity domain in that the arrhythmic waveforms like VF (and some VT) and present continuous variation of amplitude, as opposed to impulsive waveforms like normal beats and premature ventricular contractions (PVCs).

Accordingly, certain embodiments of the methodology of the present invention use the velocity domain ECG data in terms of its amplitude distribution. The methodology used is similar to multiplexing data from time sequential order to amplitude sequential order. The velocity data samples are sorted into separate groups based on velocity data sample value and contain a value equal to the number of velocity data samples within that value. Embodiments of the present invention recognize that the distribution of the groups provides information about the waveform shape that is useful for analysis. For example, the low amplitude and uniform waveforms like VF and VT will be more concentrated in the middle where as impulsive and high amplitude waveforms will be distributed in wider group ranges. This approach involves computation of the density or intensity of the velocity distribution. This intensity information is then used in conjunction with the waveform amplitude and the rate of the waveform to indicate the advisability of therapy for the best possible outcome.

Further detailed discussion of the defibrillation success predictive algorithm and AED devices and methods that implement such an algorithm are set forth in greater detail in FIGS. 1B through FIG. 6 and the following description.

In general, most AEDs have generally similar components and operate in a generally similar manner. In cases where a defibrillation shock is needed, an AED 50 may be used to deliver an impulse of high amplitude current to a patient's heart to restore it to normal cardiac rhythm. However, there are many different types of heart rhythms, only some of which are considered shockable. The primary shockable rhythms are ventricular fibrillation, ventricular tachycardia (VT), and ventricular flutter. Non-shockable rhythms may include bradycardias, electro-mechanical dissociation, idio-ventricular rhythms, and normal heart rhythms.

In order to determine if a rhythm is shockable, AEDs analyze ECG data to classify the type of rhythm the patient is experiencing. Specifically, an AED rescuer/user 60 may attach a pair of AED electrodes 104 and 106 to the chest of a cardiac arrest victim 70, as shown in FIG. 1B. The electrodes 104 and 106 communicate the ECG signal from the victim 70 to the AED 50.

AEDs relying upon such an ECG analysis may be considered semi-automatic or fully-automatic. In general, semiautomatic defibrillators require a user to press a button to deliver the actual defibrillating shock, compared to fully-automatic defibrillators that can deliver therapy without such an input of the user. Examples of such AED designs and related features can be found in U.S. Pat. Pub. No. 2011/0105930 and U.S. Pat. Nos. 5,474,574, 5,645,571, 5,749,902, 5,792,190, 5,797,969, 5,919,212, 5,999,493, 6,083,246, 6,246,907, 6,289,243, 6,658,290, 6,993,386. The disclosures of each of which is hereby incorporated by reference other than the claims or express definitions.

FIG. 1C illustrates generally a block diagram of an example ECG front end circuit 100 implementing a device configured to execute the improved initial treatment recommendation module of one embodiment of the present invention. ECG front end circuit 100 is generally implemented as a microprocessor-based system. In the ECG front end circuit 100, controller 150 coordinates the functions of the other various elements. Attached to the ECG front end circuit 100 are a pair of external electrodes 104 and 106 that can be connected across the chest of the patient 70. ECG front end circuit 100 additionally includes at least an ECG signal filter amplifier 130, a high energy delivery circuit 140, a controller 150, a memory unit 160, and a user interface 120.

In one embodiment, electrodes 104 and 106 include a copper based material and in other embodiments, electrodes 104 and 106 include other metals or materials that conduct electrical signals. When attached to a cardiac arrest victim's chest, electrodes 104 and 106 transmit electrical signals, including an ECG signal, from the victim 70 to the ECG filter and amplifier 130. Various sensors can be associated with these electrodes as well.

As noted, FIG. 1C includes an ECG signal filter and amplifier 130. Although ECG signal filter and amplifier 130 is represented by a single block in FIG. 1C, in some instances signal filter and amplifier 130 may be embodied in physically separate components. The ECG signal filter operates to largely filter out electrical signals other than ECG signals transmitted by electrodes 104 and 106. The ECG signal amplifier operates to amplify the power of the ECG signal in relation to non-ECG signals. Both the filter and amplifier can be implemented by electronic components, software techniques, or a combination of the two which are all well known in the art. After filtering and amplifying the ECG signal coming from electrodes 104 and 106, the ECG signal filter and ECG signal amplifier 130 may transmit the signal to controller 150.

Controller 150 analyzes ECG signal data and may implement the initial treatment recommendation module 180. Controller 150 receives the ECG signal data from the ECG signal filter and amplifier 130. Controller 150 may be implemented by various electronic hardware including processors, co-processors, application specific integrated circuits (ASICs), field-programmable gate arrays (FPGAs), and other electronic control circuitry well known in the art. Preferably, controller 150 includes an analog to digital converter (ADC) to digitize the ECG signal from the ECG signal filter and amplifier 130 to produce a stream of digitized ECG samples. In other embodiments, the ADC may be separate from controller 150. In even other embodiments, the ADC may convert the ECG signal into digital samples before the signal gets to the ECG signal filter and amplifier 130. Controller 150 is configured to communicate with the other various components of the ECG front end circuitry 100.

In one illustrative embodiment, recommendation module 180 may be implemented through software consisting of a set of instructions that may stored in general memory 160. In other embodiments, the instructions implementing recommendation module 180 may be stored in specialized memory associated with any of the electronic components implementing controller 150. In other embodiments, recommendation module 180 may implemented by specialized electronic circuitry including specialized processors, ASICs, FPGAs, or other electronic hardware. These electronic components may be separate from, or a part of, the electronic components implementing controller 150. In yet other embodiments, recommendation module 180 may be implemented as a combination of both hardware and software, with certain functions facilitated by hardware, and other functions facilitated by a combination of hardware and software. Ultimately, recommendation module 180 determines, or helps to determine, whether it is appropriate to deliver a high energy shock to the victim 70. When so instructed, high energy delivery circuit 140 delivers the necessary high energy shock.

High energy delivery circuit 140 is connected to both the controller 150 and the electrodes 104 and 106. The high energy delivery circuit is capable of generating and storing a large electrical potential. When commanded by the controller 150, the high energy delivery circuit 140 can transmit the generated or stored energy through the electrodes 104 and 106 and into the cardiac arrest victim 79.

Memory 160 may consist of read only memory (ROM), electrically erasable read only memory (EEPROM), random-access memory (RAM), or any other non-volatile storage medium. Memory 160 may store information, including in some embodiments, a portion of the recommendation algorithm. Controller 150 may read information from, or write information into memory 160.

Various methods utilized by embodiments of the present invention generally consist of gathering ECG data, transforming ECG data, and determining a SI number. Based at least in part on the value of the SI number, AED 50 of the present invention will issue an initial treatment recommendation to either perform CPR on the victim 70 or to deliver a high energy shock from the AED.

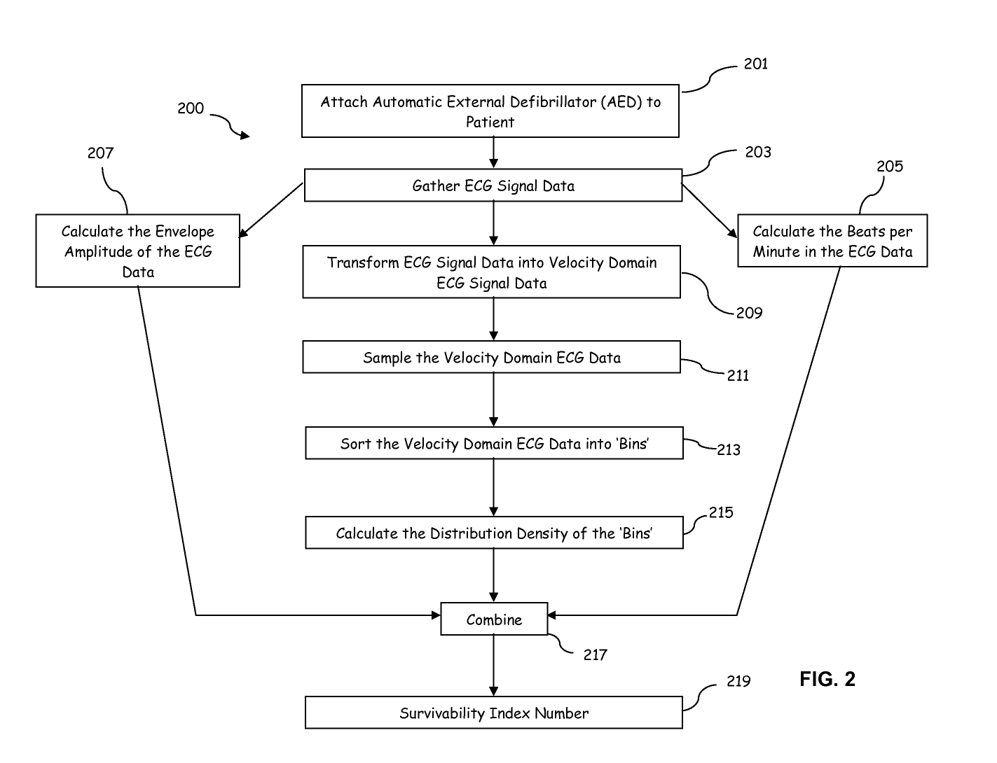

FIG. 2 sets out a more detailed flow diagram of an illustrative SI method 200, of determining an SI number. Block 201 indicates that example SI method 200 begins with attaching electrodes 104 and 106 to the chest of a cardiac arrest victim 70. Once attached to the victim 70, electrodes 104 and 106 can transmit ECG data from the victim 70 to the ECG signal filter and amplifier 130. This step is encompassed by block 203. After filtering out non-ECG signals and amplifying the ECG signal, ECG signal filter and amplifier 130 may pass the modified ECG signal to controller 150. Controller 150, either alone or in combination with recommendation module 180, transforms the data into the velocity domain (block 209). Transforming the ECG data into the velocity domain is accomplished by taking the first derivative of the incoming ECG signal data. A representative mathematical equation for performing the first derivative can be illustrated as:

$$V ( x ) = \frac{d x}{d t}$$

where

V(x): velocity domain ECG signal

x=ECG signal

In another step, illustrated by block 205, controller 150, alone or in combination with recommendation module 180, calculates the number of beats per minute present in the ECG signal. Block 207 indicates that the controller 150, alone or in combination with recommendation module 180, may also calculate the envelope amplitude of the ECG signal. The steps represented by blocks 205, 207, and 209 may be performed in any particular order or, in some embodiments, in parallel. Further, steps 207 and 205 may be performed any time before step 217.

In step 211, the ADC transforms the ECG signal into digital samples. In alternative embodiments, steps 205, 207, and 209 may be performed after the ADC digitizes the ECG signal and creates samples of the data.

In step 213, controller 150, alone or in combination with recommendation module 180, processes the digital ECG signal samples and sorts the samples in different groups or ‘bins.’ Each ‘bin’ is associated with a different sample value. Each sample is sorted into the ‘bin’ that corresponds to the value of the sample. The value stored in each ‘bin’ corresponds to the number of samples that have been sorted into that ‘bin.’

After sorting the sample into ‘bins,’ in step 215, controller 150, alone or in combination with recommendation module 180, determines a distribution density (or “intensity”) of the values stored in the ‘bins.’ The distribution density, R(v), may be represented by a mathematical equation:

R(v)=sum[DV(t)]/[max(bin-num)−min(bin-num)]

Once the distribution density has been determined, controller 150, alone or in combination with recommendation module 180, combines the distribution density, the calculated beats per minute, and the envelope amplitude of the ECG data to determine an SI number. In some embodiments, determining the SI number only involves using the distribution density. In other embodiments, combinations that include the distribution density and the calculated beats per minute, or the distribution density and the envelope amplitude of the ECG data are used to determine the SI number. In some embodiments, the SI number is scaled to be between 0 and 4 Scaling of the SI numbers and relating the numbers to corresponding recommendations may take on various parameters. For example, in some embodiments SI numbers over “1” will result in shock recommendations and SI below “1” will result in CPR recommendations. Other embodiments may make use of SI numbers scaled somewhat differently and may recommend various treatments based upon other values or ranges of values.

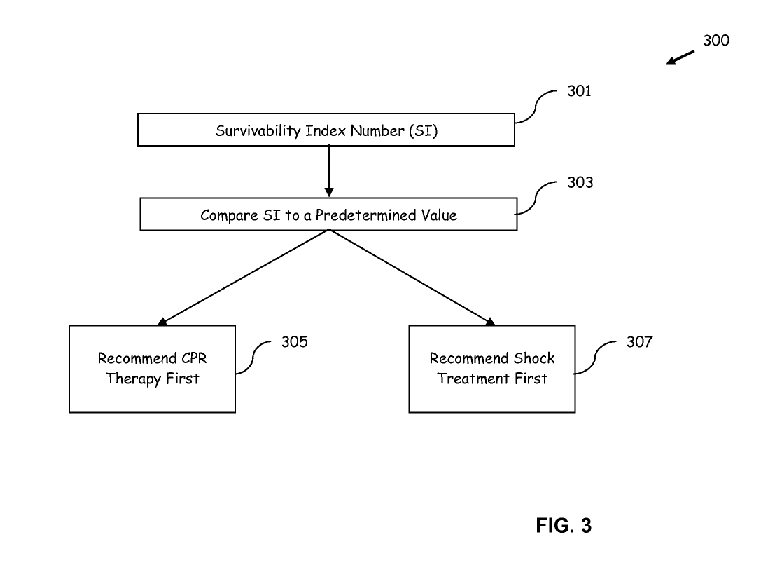

FIG. 3 illustrates a flow diagram of treatment recommendation method 300. Treatment recommendation method 300 begins by using the calculated SI number from SI method 200 and comparing the SI number to one or more pre-determined SI thresholds. In one embodiment, if the SI number is higher than a predetermined SI threshold, treatment recommendation method 300 may recommend, as an initial treatment, that the rescuer deliver a high energy shock to the victim 70 from AED 50. In some embodiments, AED 50 communicates its recommendation to the rescuer through user interface 120. In embodiments where AED 50 is fully automatic, AED 50 may administer the shock automatically without the assistance of the rescuer. In other embodiments, AED 50 may give instructions to the rescuer through the user interface 120 and wait for input from the rescuer before delivering the high energy shock. If the SI number is lower than a predetermined SI threshold, treatment recommendation method 300 may recommend the rescuer perform CPR as an initial treatment. In other embodiments, treatment recommendation method 300 may determine that CPR is the appropriate initial treatment if the SI number is higher than a predetermined value and that delivering a high energy shock is the appropriate initial treatment if the SI number is lower than a predetermined SI threshold value.

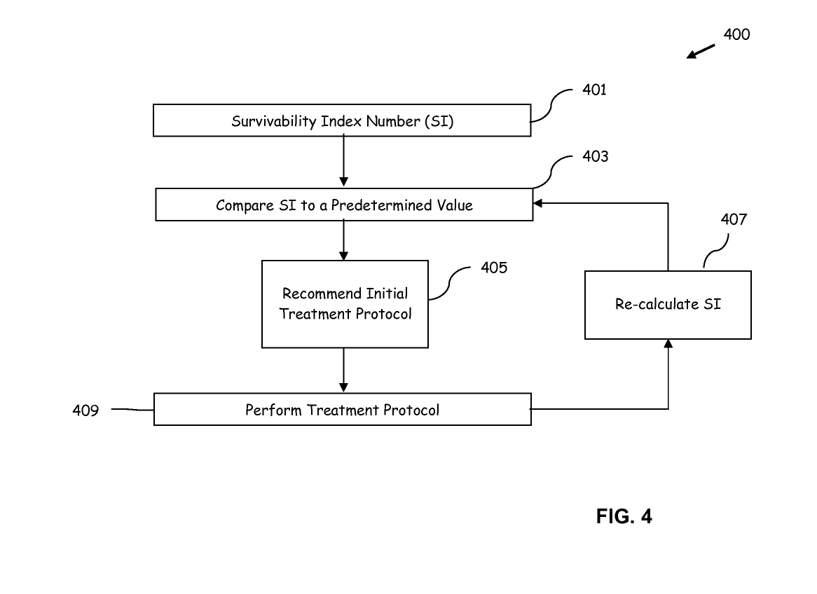

Because the SI number generally varies as a function of time, an SI number calculated during or after the initial treatment may be different than the SI number produced by treatment recommendation method 300. For example, if the initial treatment recommendation was to perform CPR, as the CPR is being performed, the SI number may change such that the new SI number would indicate that a high energy shock is now the appropriate treatment. FIG. 4 illustrates extended treatment method 400, which may be employed after treatment recommendation method 300 produces an initial treatment recommendation. Extended treatment method 400 continually updates the victim's SI number and the current appropriate treatment. Extended treatment method 400 begins, at step 401, by calculating the SI number. After comparing the SI number to predetermined SI threshold, as in recommendation method 300, extended treatment method 400 will issue a treatment recommendation. If the treatment fails to produce a normal heart rhythm, extended treatment method 400 will continue calculating SI numbers and issuing treatment recommendations. The continual updating and issuing of treatment recommendations is especially important because CPR alone is generally insufficient to restore normal electrical rhythm in a heart. As such, extended treatment method 400 allows a rescuer to know when it is appropriate to deliver a high energy shock in an attempt to restore normal heart rhythm.

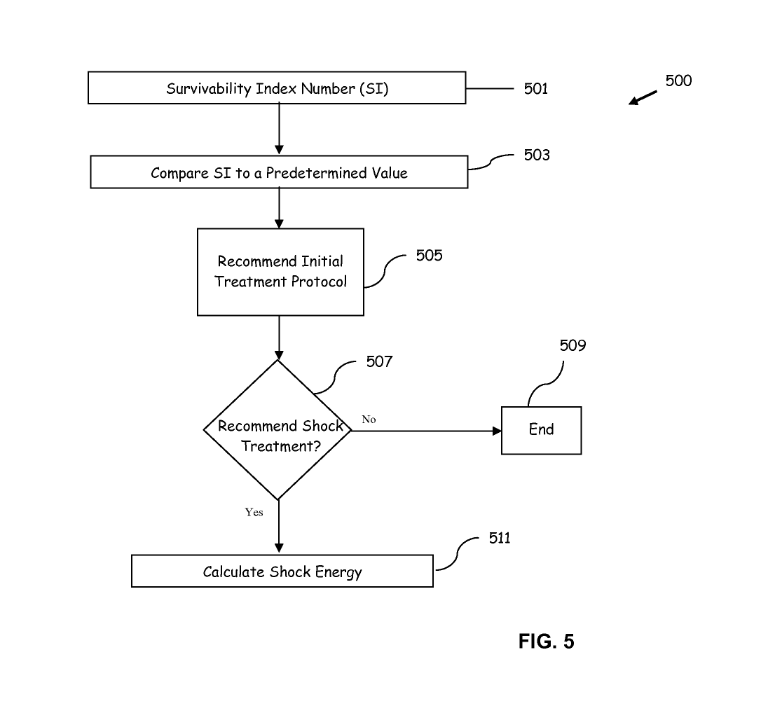

FIG. 5 demonstrates a shock energy method 500 which may be implemented along with recommendation method 300 and extended treatment method 400. Shock energy method 500, utilizing a calculated SI number, determines the appropriate level of energy with which to shock a cardiac arrest victim 70 when recommendation method 300 or extended treatment method 400 recommends delivering a shock. Steps 501, 503, and 505 are similar steps to those discussed in FIGS. 3 and 4. If the outcome of recommendation method 300 or extended treatment method 400 is to deliver a shock, as in step 507, then shock energy method 500 determines the appropriate energy level of the shock. In calculating the appropriate shock energy level in step 511, shock energy method 500 may compare the calculated SI number to predetermined SI threshold. Based on the comparison, shock energy method 500 may recommend using either a high shocking energy, a medium shocking energy, or a low shocking energy, with the recommendation designed to optimize the chance of the shock restoring a normal heart rhythm. If recommendation method 300 or extended treatment method 400 do not recommend delivering a shock, as in step 509, then shock energy method 500 does not continue on to calculate a shock energy level.

In general, it should be appreciated that the forgoing methods using a survivability index have numerous advantages. Namely, the SI allow selection of patients ready for defibrillation where the initial therapy by default is CPR. The SI may also allow selection of patients requiring CPR where the initial therapy default is defibrillation. The SI may further allow for selection of higher initial energy for defibrillation where the patient is of borderline survivability. Moreover, the SI may identify conditions in which the available protocols are inappropriate, such as in cases where electrodes are not properly attached, regular or superventricular rhythms in which values are outside a normal range for shockable VT or VF.

Further, where embodiments of the methodology of the present invention do not suffer from the disadvantages of other current methodologies that rely only on amplitude and frequency distribution of ECG data which are more prone to noise and more cumbersome to compute. Embodiments of the invention provide a consistent predictive function, monotonic with downtime, that provides a more useful predictor on both ends of the survival range than presently known devices and systems. Embodiments of the invention are further advantageous in that they provide linear behavior through mid ranges of survivability which allows a useful defibrillation energy recommendation and reduces the caps in CPR due to failed shocks at too low of an energy. Furthermore, embodiments of the disclosed design provide safety features which help prevent misapplication of data.

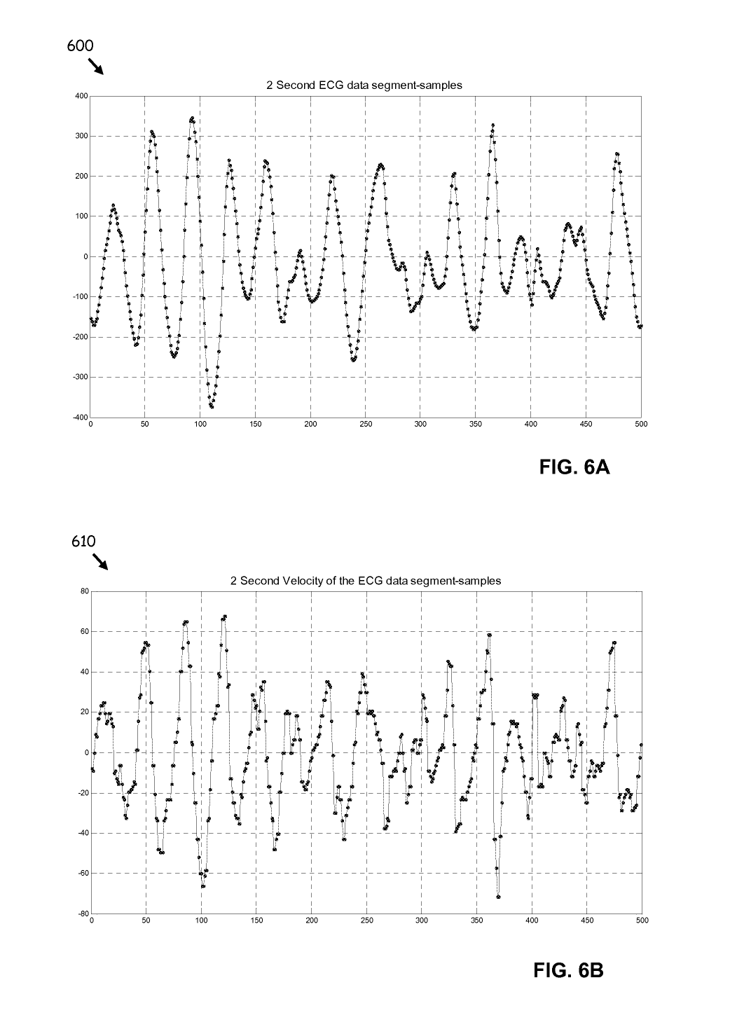

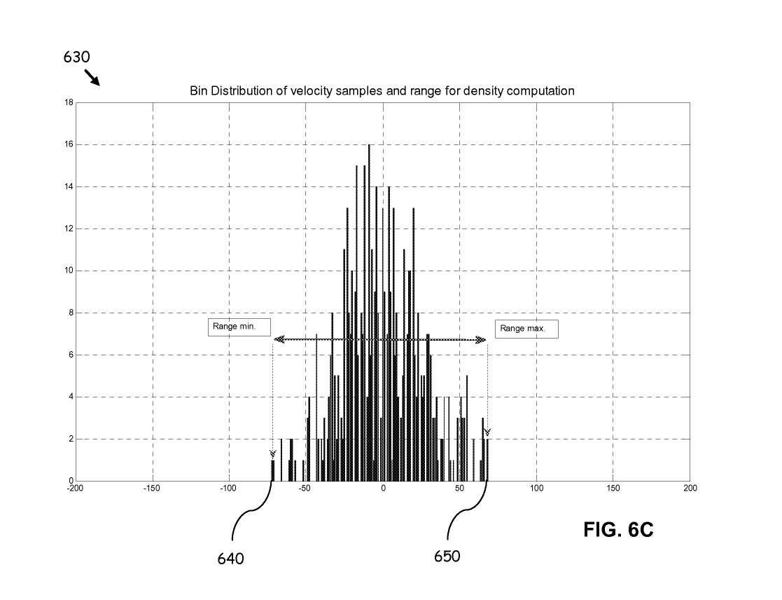

Additional illustrative figures visually demonstrating one embodiment and use of ECG data segment samples to obtain a potential SI number are shown in FIGS. 6A-C. These figures provide a better understanding of the SI parameter that is discussed in this application. In general, FIG. 6A sets forth a two second ECG data segment sample. This is reference as numeral 600. Specifically, FIG. 6A displays a 2-second ECG waveform data (Ventricular Fibrillation) segment in time domain, data sampled at the rate of 250 samples per second. The horizontal axis is in sample numbers and the vertical axis is in counts. Analysis is performed over data windows with varying lengths (1.0 sec.<window<3.0 sec.). The signal data here is Eₙ(t), for the respective “n”th discrete ECG signal samples.

FIG. 6B sets forth the two second velocity of the ECG data sample which is the velocity vector calculated from FIG. 6A. More precisely, FIG. 6B, displays the first difference “velocity” of the ECG data corresponding to the segment shown in FIG. 6A. These velocity data samples are transformed and mapped into “bins” to produce a velocity distribution. This is reference as numeral 610. Here the velocity vector calculated from FIG. 6A is shown using a continuous time domain. The velocity data here is Vn(t), for the respective “n”th discrete ECG velocity samples. Specifically, the velocity function is:

Vₙ(t)=Eₙ(t)−Eₙ₋₁(t)

Where “n” is the sample count in the discrete ECG data. This is the discrete representation of the analog derivative computation:

V(t)=∂E(t)/∂t

Next, FIG. 6C displays the distribution of the velocity converted data samples (corresponding to the data segment in FIG. 6A), into bins as such the value of the velocity determines which bin the samples belong. The density of the distribution is then computed between the maximum and the minimum range of the distribution pursuant to the velocity distribution density “Pᵥ” calculation below.

Specifically, the Velocity Distribution Density “Pᵥ” is computed as:

$${P}_{v} = \begin{matrix}Rmax \\ ( {Σα}_{i} ) / ( Rmax - Rmin + 1 ) \\ i = Rmin\end{matrix}$$

where, “Rmax”, “Rmin” are the maximum and minimum values for the distribution range, and “αᵢ” is the number of samples in a velocity distribution bin. The bin distribution of FIG. 6C is referenced by numeral 630. Rmin is represented at numeral 640 and Rmax is represented at numeral 650 in the FIG. 6B

In some embodiments, Survivability Index (SI) can then be calculated according to the following computation:

SI=100*(bps+ppr)/(sc_{—}DEN*Pᵥ)

where, “bps” is the average peak to peak wavelength in one second window, and “ppr” is peak to peak amplitude of the waveform in millivolts. The factor “sc_DEN” could have a value of “4.5” is some embodiments, for example.

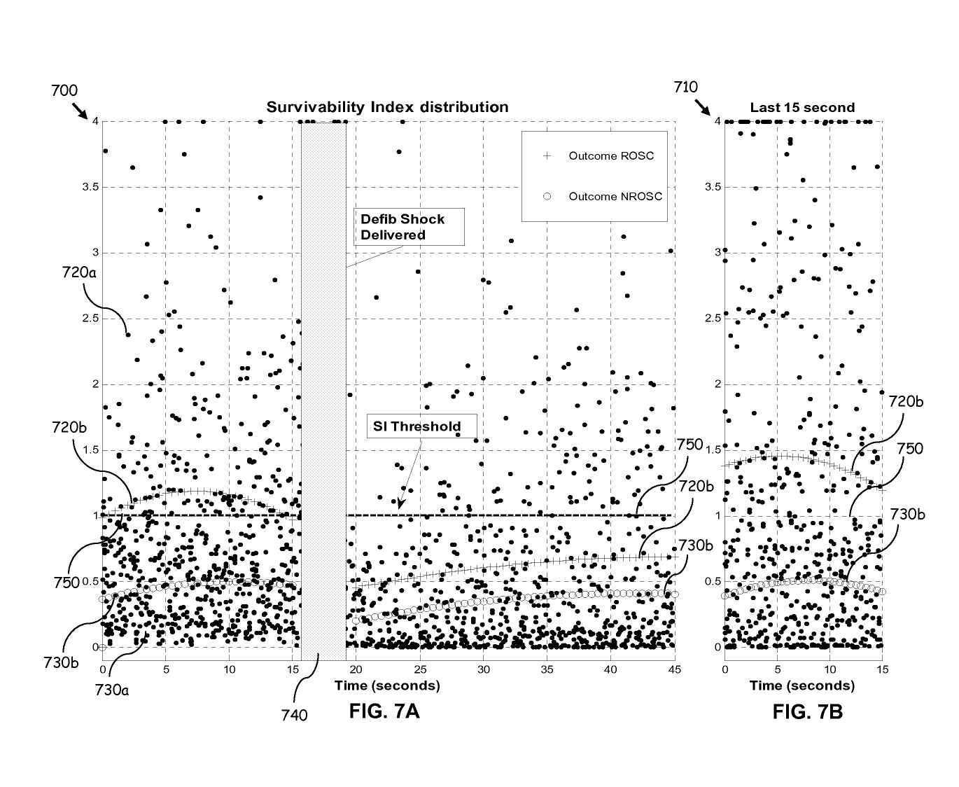

Application of the SI to patient rescue data shows that this parameter is highly effective in accessing the necessary rescue protocol for treating cardiac arrest victims. FIGS. 7A and 7B set forth graphs of the SI index values for over a variety of documented patient rescue files. The plots are respectively referred to by numerals 700 and 710. Specifically, FIGS. 7A and 7B show a display of Survivability Index (SI) values measured from several rescue files with both “ROSC” (Return Of Spontaneous Circulation) and “NROSC” (No Return Of Spontaneous Circulation) outcomes. The SI values were calculated over four windows each fifteen second long. The first three windows (forty-five seconds long) in FIG. 7A start fifteen seconds before the shock is delivered and last until thirty seconds after the shock. The second pane in FIG. 7B displays the last fifteen seconds of rescue data.

The dots 720a and their corresponding “+” curve 720b for this data represent the individuals which experienced a ROSC. The dots 730a and corresponding “o” curve 730b for this data represent the patients which did not return to a ROSC and did not survive. The vertical bar 740 represents shock delivery around which the data is matched, and the horizontal line 750 corresponding to a survivability index value of “1” represents the “survivability line”. The data in FIG. 7A shows the data surrounding the timeframe of a defibrillation shock and the data in FIG. 7B shows the data for the last 15 second of the rescue. The data generally demonstrates that patients with a higher survivability index ultimately had an increased chance of survival following a defibrillation shock, while those with a low survivability index had little change in survivability following a defibrillation shock. Accordingly, use of this SI calculation in the embodiments set forth in this disclosure and others should be considered a useful tool in providing an assessment of treatment for a cardiac arrest victim.

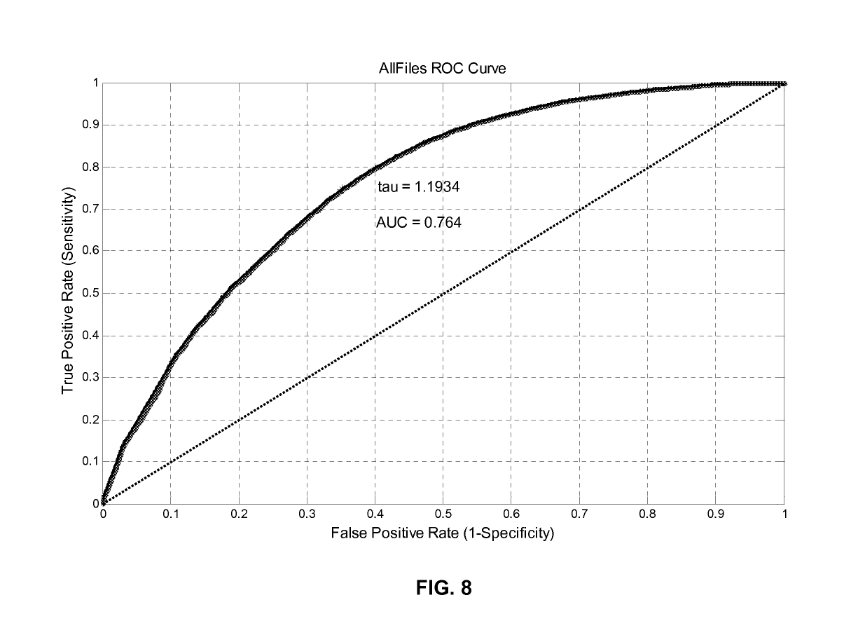

FIG. 8 shows the true and false positive response to the disclosed method as applied to 240 patient data files. Shown is a display of an ROC curve showing the sensitivity and the specificity of the disclosed method. This data revealed an AUC (area under the curve) of 0.764 with respect to correctly determining survivability rate. In general, data like this illustrates an impressive assessment of the performance of the methodology set forth and the ability to correctly determine the appropriate therapy to apply to a patient.

It should also be appreciated that the exemplary embodiment or exemplary embodiments are only examples, and are not intended to limit the scope, applicability, or configuration of the invention in any way. Rather, the foregoing detailed description will provide those skilled in the art with an enabling disclosure for implementing the exemplary embodiment or exemplary embodiments. It should be understood that various changes can be made in the function and arrangement of elements without departing from the scope of the invention as set forth in the appended claims and the legal equivalents thereof.

The embodiments above are intended to be illustrative and not limiting. Additional embodiments are within the claims. Although the present invention has been described with reference to particular embodiments, workers skilled in the art will recognize that changes may be made in form and detail without departing from the spirit and scope of the invention.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention.

For purposes of interpreting the claims for the present invention, it is expressly intended that the provisions of Section 112, sixth paragraph of 35 U.S.C. are not to be invoked unless the specific terms “means for” or “step for” are recited in a claim.

1. An automated external defibrillator (AED) with treatment protocols selected from a set of external defibrillation, cardiopulmonary resuscitation (CPR), or a combination thereof, comprising:
a power generation circuit that provides power to generate a defibrillation pulse for treating a shockable heart rhythm of a patient;
a pair of external electrodes adapted for delivery of the defibrillation pulse to the patient;
an ECG sensor that obtains an ECG signal data corresponding to heart activity of the patient;
a control system including a microprocessor configured to determine a survivability index number for the patient and recommend a treatment protocol using the AED by:
transforming the ECG signal data into first derivative velocity domain ECG signal data;
generating velocity domain ECG signal data samples from the first derivative velocity domain ECG signal data;
sorting the velocity domain ECG signal data samples into one or more groups based on a value of the velocity domain ECG signal data samples, wherein the value stored in each group corresponds to a number of velocity domain ECG signal data samples sorted into that group;
determining a distribution density of the values for the one or more groups;
determining the survivability index number based at least in part on the distribution density; and
selecting one of the treatment protocol based at least in part of the survivability index number as a selected treatment protocol; and
a communication system configured to communicate the selected treatment protocol to a user of the AED.

2. The AED of claim 1, wherein the control system further determines the survivability index number by obtaining an envelope amplitude of the ECG signal data.

3. The AED of claim 1, wherein the control system further determines the survivability index number by determining a number of beats per minute in the ECG signal data.

4. The AED of claim 2, wherein the control system further determines the survivability index number by determining a number of beats per minute in the ECG signal data.

5. The AED of claim 4, wherein the control system further determines the survivability index number by combining the distribution density, the envelope amplitude, and the number of beats per minute.

6. The AED of claim 5, wherein the treatment protocol is selected from the set consisting either of: performing CPR on the victim or delivery the defibrillation pulse to the patient with the AED.

7. The AED of claim 5, wherein the treatment protocol is a treatment to initially perform on the patient.

8. The AED of claim 5, wherein the survivability index number is repeatedly determined by the control system and the treatment protocol is repeatedly updated.

9. The AED of claim 5, wherein the control system further uses the survivability index number to determine a level of energy for the power generation circuit to use to generate the defibrillation pulse.

10. A method of automatically determining an appropriate treatment for a cardiac arrest victim with an AED, the method comprising:
obtaining ECG signal data from the cardiac arrest victim;
using a control system in the AED for:
transforming the ECG signal data into first derivative velocity domain ECG signal data;
determining a survivability index number for the cardiac arrest victim by:
generating velocity domain ECG signal data samples from the velocity domain ECG signal data;
sorting the velocity domain ECG signal data samples into one or more groups based on a value of the velocity domain ECG signal data samples, wherein the value stored for each group corresponds to a number of velocity domain ECG signal data samples sorted into that group;
determining a distribution density of the values for the one or more groups; and
determining a survivability index number based at least in part on the distribution density;
determining a selected treatment protocol for the cardiac arrest victim based at least in part on the survivability index number, wherein the selected treatment protocol is selected from a set of: external defibrillation, CPR or a combination of external defibrillation and CPR; and
using the AED to communicate the selected treatment protocol to an operator of the AED.

11. The method of claim 10, wherein determining the survivability index number further comprises obtaining an envelope amplitude of the ECG signal data.

12. The method of claim 10, wherein determining the survivability index number further comprises determining a number of beats per minute in the ECG signal data.

13. The method of claim 11, wherein determining the survivability index number further comprises determining a number of beats per minute in the ECG signal data.

14. The method of claim 13, wherein determining the survivability index number further comprises combining the distribution density, the envelope amplitude, and the number of beats per minute to generate the survivability index number.

15. The method of claim 14, wherein the selected treatment protocol comprises either performing CPR on the cardiac arrest victim or shocking the cardiac arrest victim with the AED.

16. The method of claim 14, wherein the selected treatment protocol is the treatment to initially perform on the cardiac arrest victim.

17. The method of claim 14, wherein the survivability index number is repeatedly determined and the selected treatment protocol is repeatedly updated.

18. The method of claim 14, wherein the method further comprises using the survivability index number to determine a level of energy to use for the AED to generate a defibrillation pulse.
